(19) United States
(12) Patent Application Publication
(10) Pub. No.: US 2009/0260263 A1
(43) Pub. Date: Oct. 22, 2009

(54) Toe Cover

(76) Inventor: Marcia A. Beard, Clinton Township, MI (US)

Correspondence Address: GREGORY T. ZALECKI, 12900 HALL ROAD, SUITE 400, STERLING HEIGHTS, MI, 48313, US

(21) Appl. No.: 12/106,273

(22) Filed: Apr. 19, 2008

(51) Int. Cl. A43B 13/22; A61F 5/00; A43B 23/00

(52) U.S. Cl. 36/72 R; 602/30

(57) ABSTRACT

A toe cover is comprised of a foam member, two fabric members, an adhesive strip and a nonstick cover. The fabric members cover the foam member. The foam member and the fabric members are shaped to fit between the toes and the upper surface of the shoe. The fabric members encase the foam member with serger stitching. The nonstick cover covers the adhesive strip which is positioned on top of the toe cover. The adhesive will secure the toe cover to the shoe. The toe cover prevents shoe induced toe injuries and promotes comfort. Alternate versions of the toe cover have one or more pockets. At least one side of each pocket contains a layer of foam.

### BACKGROUND

Virtually every person wears shoes on a daily basis. Many people own and wear more than one pair of shoes. Each shoe fits differently. Some shoes are comfortable to shoe wearers, others are not. Comfort is determined by shoe design, material used to fabricate the shoe, whether the shoe is broken in and the fit between the foot of the wearer and the shoe. A poorly fit or a poorly designed shoe can cause foot and toe discomfort. Walking with shoes for long distances or long periods of time can also cause discomfort. In more extreme cases foot and toe blisters, as well as other foot injuries can result from poor shoe fit, poor shoe design, wearing shoes for long periods of time or walking great distances with shoes. The toes of a person wearing shoes are particularly vulnerable to discomfort and injury. Such toe injuries even include bleeding under the toenails resulting in long-term toe nail discoloration. Athletes engaged in the performance of a sport are particularly vulnerable to foot and toe discomfort and injury.

Shoe models are at a heightened risk for foot and toe discomfort and injury. Their occupation involves displaying shoes by wearing them and walking in them. The shoe models often wear many different pairs of shoes within a short period of time. They have a need for continual foot and toe comfort and injury avoidance.

There is a need for a device to be worn upon a person's toes which will maximize comfort and minimize injury. The device should be able to be manufactured at a low cost. It should be comfortable. It should allow for quick replacement. If it is comprised of one or more pockets, each pocket should have an elastic opening.

### SUMMARY

The embodiments of toe covers described herein satisfied these needs.

A first embodiment of a toe cover is comprised of a foam member, two fabric members, an adhesive strip and a nonstick cover. The foam member can be foam rubber. The fabric members are made from cloth. One fabric member covers each side of the foam member. Serger stitching is used along the periphery of the fabric members to encase the foam member within the fabric members. An adhesive strip is attached to the top side of the fabric encased foam member. This allows the toe cover to be secured to the upper inner portion of the toe section of the shoe worn by a user. A nonstick cover covers the adhesive strip to protect the adhesive strip before the toe cover is used and to prevent unintended bonding of the toe cover. The foam member and the fabric members are shaped to fit between the upper surface of the toes and the upper inner portion of the toe section of a shoe worn by a user. The toe cover may be oval, rectangular, pear-shaped or shaped in another configuration, so long as it fits between the upper surface of the toes and the upper inner portion of the toe section of a shoe. The toe cover should be shaped to conform to the shape of the upper inner portion of the toe section of the shoe within which it is intended to be used. If the intended shoe to be used is an open toed shoe, the toe cover should be shaped such that it is confined to the upper inner portion of the toe section of the shoe when the shoe is worn by a user so that the toe cover does not extend into the open toe area of the shoe. For increased comfort, the serger stitching should be comprised of elastic thread and have a stitch to stitch width of less than 1.1 mm.

A second embodiment of a toe cover is comprised of a foam member, a first fabric member, a second fabric member and a third fabric member. The foam member can be foam rubber. The fabric members are made from cloth. The first fabric member and the second fabric member encase the foam member within those two fabric members. The third fabric member is attached to the first fabric member, the second fabric member and the foam member such that a pocket is formed for receiving the toes of a user. The pocket is formed between the third fabric member and one of the other fabric members. The foam member and the fabric members are shaped to fit within the inner portion of the toe section of a shoe worn by the user. The first fabric member should be attached to the second fabric member by serger stitching at the opening of the pocket. The first fabric member and the second fabric member encase the foam member to form a pad. The third fabric member is attached to the first fabric member, the second fabric member and the foam member by serger stitching. The serger stitching should be comprised of elastic thread with a stitch to stitch width of less than 1.1 mm.

A third embodiment of a toe cover is comprised of a foam member and two fabric members. The foam member can be foam rubber. The fabric members are made from cloth. One fabric member covers each side of the foam member. The foam member and the fabric members are formed into a pocket for receiving the toes of the user. The foam member and the fabric members which are formed into the pocket are shaped to fit within the upper inner portion of the toe section of a shoe worn by the user. The fabric covered foam formed into the pocket should be attached to itself at the sides of the pocket by serger stitching on the interior aspect of the pocket. All other stitching should also be serger stitching. The serger stitching should be comprised of elastic thread with a stitch to stitch width of less than 1.1 mm. Preferably, all serger stitching used in any of the embodiments should be oriented so that any stitching which contacts the toes or foot of a user has contact made at the finished edge of the serger stitching.

A fourth embodiment of a toe cover is comprised of a foam member and three fabric members. The foam member can be foam rubber. The fabric members are made from cloth. The first fabric member covers one side of the foam member. The second fabric member covers the other side of the foam member. The third fabric member covers the first fabric member. The foam member and the fabric members are formed into a configuration having three fabric lined pockets. Each of the pockets is adapted to receive the toes of a user. The first of the pockets has a portion of the foam member above and another portion of the foam member below the pocket opening. The second of the pockets has the first pocket and the third pocket on the upper side of the second pocket. The third pocket has the first pocket and the second pocket on the lower side of the third pocket. The first pocket is positioned between the second pocket and the third pocket. The upper layer and the lower layer of the three pocket toe cover consists of fabric only. In other words the upper layer and the lower layer of the fabric do not encase the foam member. The foam member and the fabric members which are formed into the pockets are shaped to fit within the inner portion of the toe section of a shoe worn by the user. The fabric covered foam formed into the pockets should be attached to itself at the sides of the pockets by serger stitching on the interior aspects of the pockets. The serger stitching should be comprised of elastic thread with a stitch to stitch width of less than 1.1 mm.

### DRAWINGS

These and other features, aspects, and advantages of the present invention will become better understood with regard to the following description, appended claims, and accompanying drawings where:

FIG. 1 is a perspective view of a first embodiment of a toe cover being worn by a user.

FIG. 2 is a perspective view of a first embodiment of a toe cover.

FIG. 3 is a top plan view of the toe cover of FIG. 2.

FIG. 4 is an elevational sectional view of the toe cover of FIG. 3.

FIG. 5 is an enlarged partial elevational sectional view of the toe cover of FIG. 4.

FIG. 6 is a top plan view of a second embodiment of a toe cover.

FIG. 7 is a front elevational sectional view of the toe cover of FIG. 6.

FIG. 8 is a front elevational sectional view of a third embodiment of a toe cover.

### DESCRIPTION

A toe cover 26 is intended to be used within a shoe 20. The shoe 20 has an upper inner portion of a toe section 22. This is where the toes 24 of a user interface with the shoe 20.

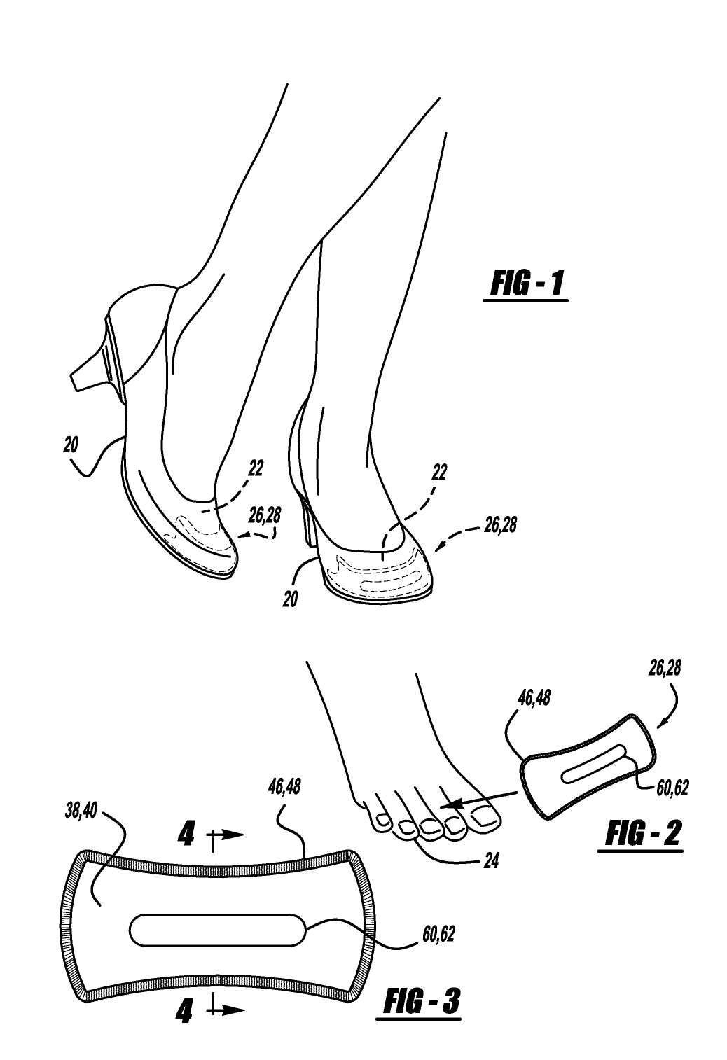

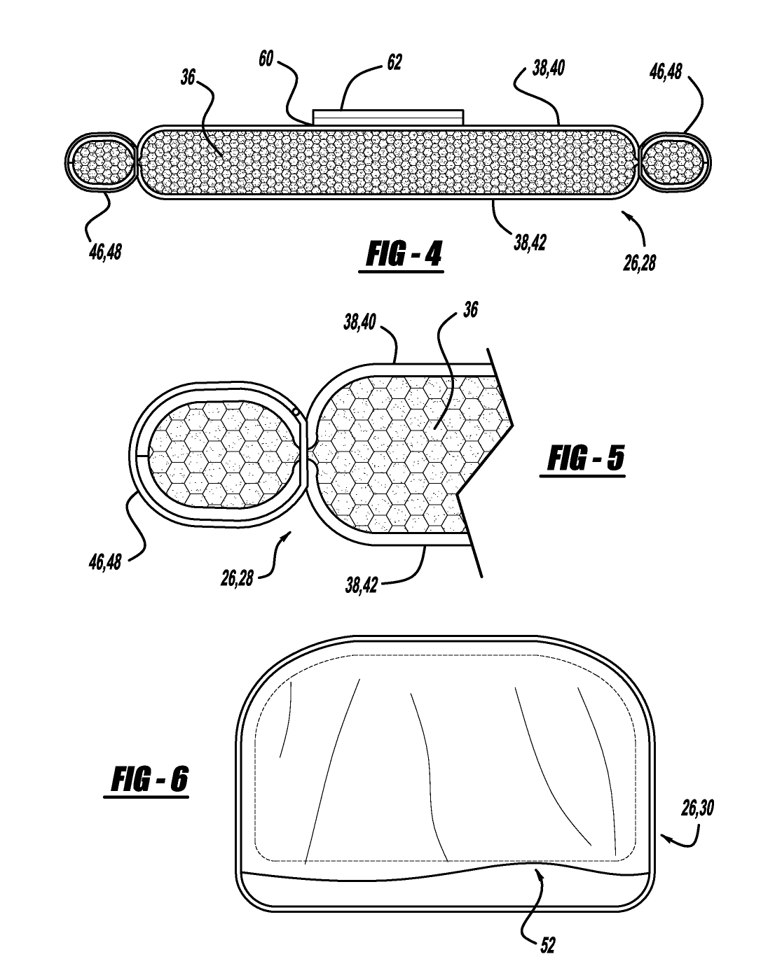

A first embodiment of a toe cover 26, 28 is shown in FIG. 1 through FIG. 5. This toe cover 26, 28 is comprised of a foam member 36, two fabric members 38, 40, 42, an adhesive strip 60 and a nonstick cover 62. The preferred foam of the foam member 36 is 80 PPI (pores per inch) natural polyester foam. The term foam includes light, porous, semi rigid and spongy materials used for shock absorption and other materials commonly referred to as foam. One fabric member 38, 40 covers the top side of the foam member 36. The other fabric member 38, 42 covers the bottom side of the foam member 36. Serger stitching 46 along the periphery of the fabric members 38 encase the foam member 36 within the fabric members 38.

Preferably, the edges of the foam member 36 are entrapped by the serger stitching 46. This is shown in FIG. 4 and FIG. 5. The edge entrapped foam surrounded by serger stitching 46 provides an added degree of comfort to the user. Some of these areas will ultimately contact the toes 24 of the user. The entrapped foam member 36 provides cushioning. The serger stitching 46 provides a smooth covering. Whenever other embodiments of toe covers 26 described herein employee fabric covered foam, the described edge entrapped foam surrounded by serger stitching 46 is also preferred.

A serger is a special-purpose machine that produces professionally finished seams, like those found on ready-to-wear garments. It uses between two and nearly a dozen threads to encase the raw edge of the fabric with an overcast stitch, while trimming away the seam allowance. This can be done at speeds approaching 1700 stitches per minute. Instead of a bobbin and single needle, the serger has a cutting blade, loopers and multiple needles. Segers are used to clean finish seam allowances. The primary function of a serger is to clean finish all raw edges, giving a professional appearance. There are many varieties of serger stitches. The term serger stitching as used herein includes all the varieties. In general, serger stitches are thread spirals extending along an interior longitudinal axis. The use of serger stitching to attach the fabric members 38 within the toe covers 26 allows the edge of a foam member 36 to be encased by the serger stitching 46, as shown in FIG. 4 and FIG. 5. The encased foam areas are typically in areas of a toe cover 26 which contact the toes 24 of a user. Thus, instead of the toes 24 contacting a rough unfinished edge, they contact encased foam, thereby providing an enhanced degree of comfort. Additional comfort is provided by serger stitching 46 because the serger thread 48 isolates the edges of the fabric members 38 from the user's toes. Preferably, the stitch width is less than 1.1 mm to maximize this isolation. Stitch width is the minimum distance between adjacent stitches of serger stitching 46. Thus, with minimal stitch width the edges of the fabric members 38 can be completely isolated from the toes 24 of a user, thereby providing maximal comfort and minimal friction between the toes 24 and the toe cover 26. Therefore, not only is comfort enhanced, but the toe cover 26 is more likely to prevent injuries, such as blisters, caused by friction between the toes 24 and the upper inner portion of the toe section 22 of the shoe 20. Preferably, the thread 48 used for the serger stitching 46 is elastic thread 48. This also enhances comfort and minimizes injury potential. Further, the serger stitching 46 itself becomes elastic. In pocketed versions of the toe cover 26, the elasticity of the serger stitching 46 permits a pocket design which will limit the ability of a toe cover 26 to slip off of the user's toes 24. In such designs the pocket is adapted to lightly grasp the toes 24. One preferred version of thread 48 is sold under the trade name WOOLLY NYLON. While an entrapped foam structure has been described herein, it should be clear that entrapped foam is optional and not required. This description of serger stitching and its variations for use is intended to apply to all toe cover 26 embodiments described herein and not simply the first embodiment described.

An adhesive strip 60 is positioned on top of the toe cover 26, 28, as shown in FIGS. 1-4. The adhesive is selected to provide for the releasable attachment of the toe cover 26, 28 to the upper inner portion of the toe section 22 of the shoe 20, as shown in FIG. 1. The adhesive strip 60 is covered by a nonstick cover 62. This protects the adhesive strip 60 from inadvertent attachment to other objects before use.

The foam member 36 and the fabric members 38 are shaped to fit between the upper surface of the toes 24 and the upper inner portion of the toe section 22 of a shoe 20. This is shown in FIG. 1 and FIG. 2. The toe cover 26, 28 should be shaped to conform to the shape of the upper inner portion of the toe section of the shoe 20. The shape can be rectangular, oval, pear-shaped or custom fit to a particular shoe. Other shapes can also be used. The pear-shaped design is useful for pointed toe shoes. The longitudinal axis of the pear-shaped toe cover 26, 28 is substantially parallel to the longitudinal axis of the shoe 20 when installed. A rectangular shape modified such that the long edges are slightly arced is particularly useful for an open toed shoe. The toe cover 26, 28 is confined to the upper inner portion of the toe section 22 of the shoe 20 when the shoe 20 is worn by a user. This shape variation is such that the toe cover 26, 28 does not extend into the open toe area of the shoe 20. Rather, it remains hidden from view when worn.

The following steps describe the normal use of the first embodiment of a toe cover 26, 28. A toe cover 26, 28 is selected. The nonstick cover 62 is removed from the adhesive strip 60. The toe cover 26, 28 is attached to the upper inner portion of the toe section 22 of the shoe 20 with the adhesive of the adhesive strip 60. The user inserts his or her foot into the shoe 20. The user's toes 24 fit under the toe cover 26, 28, as shown in FIG. 1. When the user is done using the toe cover 26, 28, the toe cover 26, 28 is peeled away from the upper inner portion of the toe section 22 of the shoe 20 and disposed.

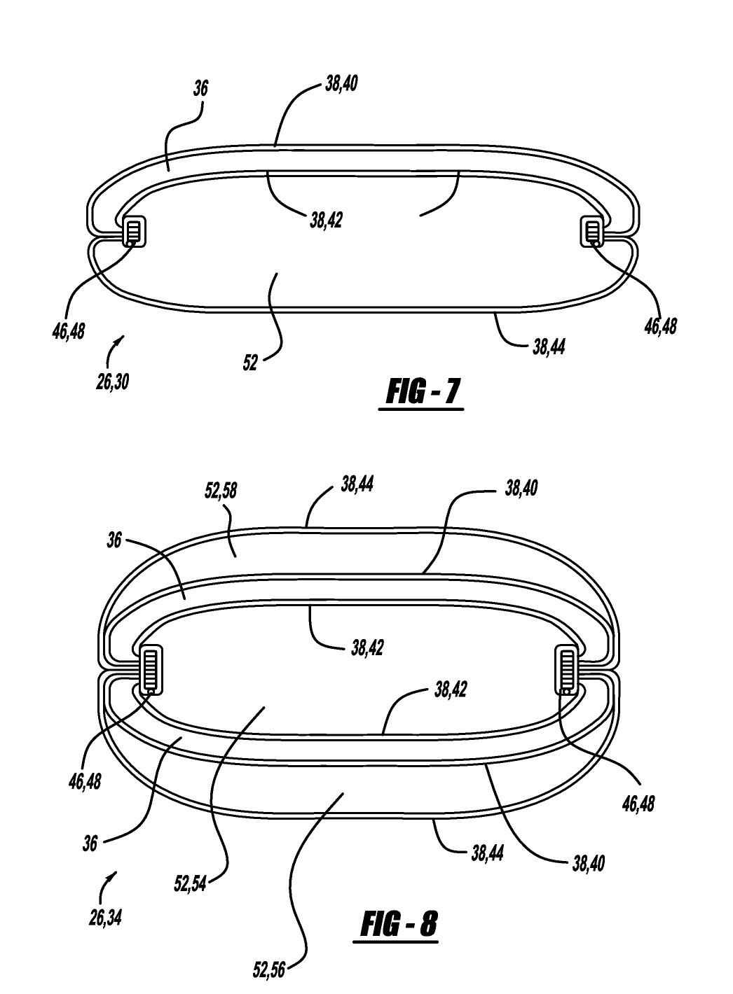

A second embodiment of a toe cover 26, 30 is shown in FIG. 6 and FIG. 7. This embodiment is comprised of a foam member 36, a first fabric member 38, 40, a second fabric member 38, 42 and a third fabric member 38, 44. The first fabric member 38, 40 and the second fabric member 38, 42 encase the foam member 36 within the first fabric member 38, 40 and the second fabric member 38, 42. Serger stitching should be used to attach the first fabric member 38, 40 to the second fabric member 38, 42. The third fabric member 38, 44 is attached to the first fabric member 38, 40, the second fabric member 38, 42 and the foam member 36 such that a pocket 52 is formed for receiving the toes 24 of the user. This is shown in FIG. 6 and FIG. 7. The foam member 36 and the fabric members 38 are shaped to fit within the inner portion of the toe section of a shoe worn by the user. The first fabric member 38, 40 should be attached to the second fabric member 38, 44 by serger stitching 46, including serger stitching 46 at the opening of the pocket 52. The third fabric member 38, 44 should be attached to the first fabric member 38, 40, the second fabric member 38, 42 and the foam member 36 by serger stitching 46. The serger stitching 46 should have a stitch to stitch width of less than 1.1 mm. The serger stitching 46 should be comprised of elastic thread 48.

The following steps describe the normal use of the second embodiment of a toe cover 26, 30. A toe cover 26, 30 is selected. The user inserts his or her toes 24 into the pocket 52 of the toe cover 26, 28. The foam member 36 should be on top of the toes 24. However, the toe cover 26, 30 can also be used with the foam member 36 on the bottom of the toes 24. The user inserts his or her foot, with the toe cover 26, 30 covering the toes 24, into the shoe 20.

FIG. 8 can be used to understand the third and fourth embodiments of this invention. The third embodiment has only one pocket 52, while the fourth embodiment has three pockets 52, 54, 56, 58. The middle section of the fourth embodiment is similar to the third embodiment.

The third toe cover 26 embodiment is comprised of a foam member 36 and two fabric members 38, 40, 42. One fabric member 38 covers each side of the foam member 36. The foam member 36 and the fabric members 38 are formed into a pocket 52. The pocket 52 is shaped to receive the toes 24 of the user. The pocket 52 is formed by attaching the foam member 36 and the fabric members 38 together at two sides. In other words, a flat one-dimensional configuration consisting of the foam member 36 encased by the fabric members 38 is first fabricated. Then, this flat one-dimensional configuration is folded onto itself to create the pocket 52. The foam member 36 and the fabric members 38 which are formed into the pocket 52 are shaped to fit within the inner portion of the toe section of a shoe worn by a user. The fabric covered foam 36 formed into the pocket 52 is attached to itself at the sides of the pocket by serger stitching 46 on the interior aspect of the pocket 52. The serger stitching 46 should be comprised of elastic thread. This will enhance comfort to the user by preventing loose edges of the fabric members 38 from contacting the toes 24 of the user. The stitch to stitch width of the serger stitching 46 should be less than 1.1 mm.

The following steps describe the normal use of the third embodiment of a toe cover 26. A toe cover 26 is selected. The user inserts his or her toes 24 into the pocket 52 of the toe cover 26. This results in a part of the foam member 36 being on top of the toes 24 and another part of the foam member 36 being below the toes 24. The pocket 52 should have an elastic quality which allows the pocket 52 to snugly fit around the toes 24. The user then inserts his or her foot, with the toe cover 26 covering the toes 24, into the shoe 20.

The fourth embodiment of a toe cover 26, 34 is shown in FIG. 8. This embodiment is comprised of a foam member 36, a first fabric member 38, 40, a second fabric member 38, 42 and a third fabric member 38, 44. The first fabric member 38, 40 covers one side of the foam member 36. The second fabric member 38, 42 covers the other side of the foam member 36. The third fabric member 38, 44 covers the first fabric member 38, 40. The foam member 36 and the fabric members 38, 40, 42, 44 are formed into a configuration having three fabric lined pockets 52, 54, 56, 58. Each of the pockets 52, 54, 56, 58 are adapted to receive the toes 24 of a user. The first pocket 52, 54 has a portion of the foam member 36 above and another portion of the foam member 36 below the first pocket 52, 54. The second pocket 52, 56 has the first pocket 52, 54 and the third pocket 52, 58 on the upper side of the second pocket 52, 56. The third pocket 52, 58 has the first pocket 52, 54 and the second pocket 52, 56 on the lower side of the third pocket 52, 58. The first pocket 52, 54 is positioned between the second pocket 52, 56 and the third pocket 52, 58. This fabrication may be accomplished by using the same technique described for fabricating the third embodiment. A flat configuration similar to that shown in the upper half of FIG. 8 is first constructed. This flat configuration is then folded upon itself to form the three pocket symmetrical configuration shown in FIG. 8. The foam member 36 and the fabric members 38, 40, 42, 44 which are formed into the pockets are shaped to fit within the inner portion of the toe section of a shoe 20 warned by a user. In other words, the embodiment shown in FIG. 8 is shaped to fit within the inner portion of the toe section of the shoe 20. The fabric 38 covered foam 36 formed into the pockets 52 is attached to itself at the sides of the pockets 52, 54, 56, 58 by serger stitching 46 on the interior aspects of the pockets 52, 54, 56, 58, as shown in FIG. 8. FIG. 8 (as well as FIG. 7 pertaining to the third embodiment) shows serger stitching 46 on the inner aspect of the first pocket 52, 54. Only the first stitch 46 on each side is visible in the drawing. The other stitches 46 on each side are longitudinally aligned behind the first stitch 46. In other words, a series of stitches should be visualized behind the first stitch 46 directed along a longitudinal axis perpendicular to the drawing sheet upon which FIG. 8 is printed. The preferred approach for making such stitches 46 is to turn the first pocket 52, 54 inside out prior to applying the serger stitching 46. In this inside out configuration the serger stitches 46 shown in FIG. 8 and FIG. 7 would be flipped so that they are temporarily positioned on the outer periphery of the toe cover 28. With the material edges on the outer periphery, application of the serger stitching is quick and easy. Upon completion of the serger stitching the toe cover 28 is manipulated by another inside out movement so that the serger stitches 46 lie within the pocket 52, as shown in FIG. 7 and FIG. 8. This prevents the edges of the fabric members 38 from directly contacting the toes 24 or foot of the user and thereby enhances comfort. The serger stitching 46 should be comprised of elastic thread and have a stitch to stitch with of less than 1.1 mm. This reduces further the possibility of fabric member 38 edges making contact with the foot or toes 24 of the user. A portion of the foam member 36 can be encased within the serger stitching 46 for added comfort.

The following steps describe the use of the fourth embodiment of a toe cover 26, 34. A toe cover 26, 34 is selected. The user inserts his or her toes 24 into the first pocket 52, 54 of the toe cover 26, 34. This is the middle pocket 52 of FIG. 8. The user then has a part of the foam member 36 on top of the toes 24 and another part of the foam member 36 on the bottom of the toes 24. The first pocket 52, 54 should have an elastic quality which allows the pocket 52 to snugly fit around the toes 24. The user inserts his or her foot, with the toe cover 26, 34 covering the toes 24, into the shoe 20. Alternatively, the toes 24 may be inserted into the second pocket 52, 56 (i.e. the bottom pocket) or the third pocket 52, 58 (i.e. the top pocket). This allows the user to have a double layer of foam 36 on the top or on the bottom of the toes 24.

Although the invention has been shown and described with reference to certain preferred embodiments and methods, those skilled in the art undoubtedly will find alternative embodiments and methods obvious after reading this disclosure. With this in mind, the following claims are intended to define the scope of protection to be afforded the inventor, and those claims shall be deemed to include equivalent constructions insofar as they do not depart from the spirit and scope of the present invention.

What is claimed is:

1. A toe cover for use within a shoe having an upper inner portion of a toe section covering the toes of a user, said toe cover comprising:
(a) a foam member;
(b) two fabric members, one fabric member covering each side of the foam member;
(c) serger stitching along the periphery of the fabric members, said serger stitching encasing the foam member within the fabric members;
(d) an adhesive strip attached to a top side of the fabric encased foam member for securing the toe cover to the upper inner portion of the toe section of the shoe; and
(e) a nonstick cover covering the adhesive strip;
(f) wherein the foam member and the fabric members are shaped to fit between the upper surface of the toes and the upper inner portion of the toe section of a shoe worn by a user.

2. The toe cover of claim 1, wherein the toe cover is shaped to conform to the shape of the upper inner portion of the toe section of the shoe.

3. The toe cover of claim 1, wherein the shoe is an open toed shoe and the toe cover is shaped such that it is confined to the upper inner portion of the toe section of the shoe when the shoe is worn by a user, whereby the toe cover does not extend into the open toe area of the shoe.

4. The toe cover of claim 1, wherein the foam is 80 PPI natural polyester foam.

5. The toe cover of claim 1, wherein the stitch to stitch width of the serger stitching is less than 1.1 mm.

6. The toe cover of claim 1, wherein the stitching is comprised of elastic thread.

7. A toe cover for use within a shoe having an inner portion of a toe section covering the toes of a user, said toe cover comprising:
(a) a foam member;
(b) a first fabric member and a second fabric member encasing the foam member within the first fabric member and the second fabric member; and
(c) a third fabric member attached to the first fabric member, the second fabric member and the foam member such that a pocket is formed for receiving the toes of the user;
(d) wherein the foam member and the fabric members are shaped to fit within the inner portion of the toe section of a shoe worn by a user.

8. The toe cover of claim 7, wherein the first fabric member is attached to the second fabric member by serger stitching at the opening of the pocket and wherein the third fabric member is attached to the first fabric member, the second fabric member and the foam member by serger stitching.

9. The toe cover of claim 7, wherein the stitch to stitch width of the serger stitching is less than 1.1 mm.

10. The toe cover of claim 7, wherein the stitching is comprised of elastic thread.

11. A toe cover for use within a shoe having an inner portion of a toe section covering the toes of a user, said toe cover comprising:
(a) a foam a member;
(b) two fabric members, one fabric member covering each side of the foam member;
(c) wherein the foam member and the fabric members are formed into a pocket for receiving the toes of the user; and
(d) wherein the foam member and the fabric members which are formed into the pocket are shaped to fit within the inner portion of the toe section of a shoe worn by a user.

12. The toe cover of claim 11, wherein the fabric covered foam formed into a pocket is attached to itself at the sides of the pocket by serger stitching on the interior aspect of the pocket.

13. The toe cover of claim 11, wherein the stitch to stitch width of the serger stitching is less than 1.1 mm.

14. The toe cover of claim 11, wherein the stitching is comprised of elastic thread.

15. A toe cover for use within a shoe having an inner portion of a toe section covering the toes of a user, said toe cover comprising:
(a) a foam a member;
(b) three fabric members;
(c) the first fabric member covering one side of the foam member and the second fabric member covering the other side of the foam member;
(d) the third fabric member covering the first fabric member;
(e) wherein the foam member and the fabric members are formed into a configuration having three fabric lined pockets, each of said pockets being adapted for receiving the toes of a user, the first of said pockets having a portion of the foam member above and another portion of the foam member below the pocket, the second of said pockets having the first pocket and the third pocket on upper side of the second pocket and the third of said pockets having the first pocket and the second pocket on the lower side of the third pocket, said first pocket being positioned between said second pocket and said third pocket; and
(f) wherein the foam member and the fabric members which are formed into the pockets are shaped to fit within the inner portion of the toe section of a shoe worn by a user.

16. The toe cover of claim 15, wherein the fabric covered foam formed into the pockets is attached to itself at the sides of the pockets by serger stitching on the interior aspects of the pockets.

17. The toe cover of claim 15, wherein the stitch to stitch width of the serger stitching is less than 1.1 mm.

18. The toe cover of claim 15, wherein the stitching is comprised of elastic thread.

19. The toe cover of claim 1, wherein a portion of the foam member is encased within the serger stitching.
